(19) United States
(12) Patent Application Publication
(10) Pub. No.: US 2023/0092766 A1
(43) Pub. Date: Mar. 23, 2023

(54) SYSTEMS AND METHODS FOR VISUALIZATION OF A TREATMENT PROGRESS

(71) Applicant: Seebe, Inc., Menlo Park, CA (US)

(72) Inventors: Kailas Vodrahalli, Los Altos, CA (US); Justin Ko, Menlo Park, CA (US); Arash Mostaghimi, Newton, MA (US)

(21) Appl. No.: 17/948,018

(22) Filed: Sep. 19, 2022

Related U.S. Application Data
(60) Provisional application No. 63/246,151, filed on Sep. 20, 2021.

(51) Int. Cl. G16H 30/40; G16H 50/50; G06T 5/00; G06T 7/00

(52) U.S. Cl. CPC G16H 30/40; G16H 50/50; G06T 5/005; G06T 7/0016; G06T 2207/30196

(57) ABSTRACT

The present disclosure provides methods and systems for predicting future disease states of a subject. An exemplary method comprises: (a) obtaining an image from an imaging device, wherein the image indicates a disease associated with the subject; (b) generating an annotation mask for a current disease state; (c) processing the image data and the annotation mask to generate a temporal sequence of images with corresponding predicted disease states; and (d) outputting the temporal sequence of images and disease analytics within a graphical user interface (GUI).

### REFERENCE

This application claims priority to U.S. Provisional Patent Application No. 63/246,151, filed on Sep. 20, 2021, which is entirely incorporated herein by reference.

### BACKGROUND

Many treatments or products may require sustained use for a period of time before results are perceptible to a user. As a result, users or patients may prematurely cease use of an effective treatment. For example, after a skin treatment has subtly diminished an appearance of acne, rashes, or the like on the user's facial skin, but before improvement is visibly perceptible to the user, a user may prematurely terminate the treatment out of impatience or a perception that the product is ineffective. Further, prognosis can be made on the basis of the normal course of the diagnosed disease, the available treatments, the individual's physical or mental condition, and/or additional factors. Allowing patients or customers to visualize a prognostic result or treatment outcome of a medical condition may assist in general education and/or counseling. For example, cosmetic surgeons and beauticians may use “before” and “after” pictures to demonstrate the apparent effect a treatment may have on a person. However, current prognostic methods and systems may lack the capability to provide accurate and personalized visualization of future progress of a disease or outcomes over a course of treatment. Hence, there are needs for improved methods and systems for prognosticating the visible effects of particular treatment protocols over time.

### SUMMARY

Recognized herein is a need for methods and systems for improved prognostic visualization or visualization of a treatment progress. The present disclosure provides methods and systems for predicting future disease state or a health-related progress and allowing for visualizing such future disease state or progression. Methods and systems of the present disclosure may create a data-driven, personalized visualization tool allowing users to visualize prognostic analytics thereby encouraging users to track and adhere to a treatment path. Methods and systems provided herein may be capable of accounting for the variability among subjects, continuously adapt to each subject over time, and/or identifying deviations from a treatment path to provide real-time actionable intervention and/or modification of the treatment.

Systems and methods herein may be used for visualizing a predicted progress of health-related condition, predicted disease progression, and/or predicted outcome of treatments over a future course of treatment. Systems and methods herein may be applicable for any medical and/or cosmetic treatment protocol where visual indicators are important for tracking the progress of the treatment, such as conditions of the skin, the eyes, the mouth, the dentition, mucous membranes, hair, and the like. The treatments may comprise, for example, treatments for dermatological condition or skin condition (e.g., acne, rosacea, vitiligo, melasma, tattoo removal, eczema, psoriasis, skin cancer, laser/cosmetics, etc.), hair loss, teeth whitening, tooth and hair coloring, weight loss, skin firmness and elasticity, skin lightening, whitening, skin blemishes and freckles, tanning, aesthetic-related conditions of the user and other biological conditions of the user. Methods and systems of the present disclosure may be implemented on a variety of platforms, including existing devices (e.g., a user's electronic device, such as a mobile device, user's wearable device, computing device, etc.).

The provided methods and systems may be capable of accounting for the variability among users (e.g., patients), real-time conditions and data, and/or continually improve without relying on supervised features (e.g., labeled data). Systems and methods of the present disclosure may utilize unsupervised learning or semi-supervised learning, and/or transfer learning for disease progression prediction and visualization based on disease specific data and user specific-data in an automated fashion.

In an aspect, a method for predicting future disease states of a subject is provided. The method may comprise: (a) obtaining an image from an imaging device, where the image indicates a disease associated with the subject; (b) generating an annotation mask for a current disease state; (c) processing the image data and the annotation mask to generate a temporal sequence of images with corresponding predicted disease states; and (d) outputting the temporal sequence of images and disease analytics within a graphical user interface (GUI).

In some embodiments, the disease analytics comprise scores of the future disease states over a course of treatment. In some embodiments, generating the annotation mask comprises receiving a user input within the GUI. In some cases, the user input indicates a general region of interest or an area with the disease on the image data displayed on the GUI.

In some embodiments, the method further comprises receiving a user input indicating a selection of a treatment. In some cases, generating the temporal sequence of images with corresponding predicted disease states comprises using an inpainting model to generate an image without the disease, and wherein the image without the disease corresponds to a final disease state. In some instances, the method further comprises generating a first image embedding corresponding to the current disease state and a second image embedding corresponding to the final state as an outcome of the treatment. For example, the method may further comprise generating one or more temporal image embeddings corresponding to one or more disease states between the current disease state and the final state.

In some cases, an encoder of the inpainting model is used as an image embedding model to generate the one or more temporal image embeddings. In some cases, the inpainting model comprises an encoder and decoder formed by gated convolution.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein. In some embodiments, a system for predicting future disease states of a subject is provided. The system comprises: (i) a memory for storing a set of software instructions, and (ii) one or more processors configured to execute the set of software instructions to: (a) receive an image acquired by an imaging device, where the image indicates a disease associated with the subject; (b) generate an annotation mask for a current disease state; (c) process the image data and the annotation mask to generate a temporal sequence of images with corresponding predicted disease states; and (d) output the temporal sequence of images and disease analytics within a graphical user interface (GUI).

In some cases, the disease analytics comprise scores of the future disease states over a course of treatment. In some embodiments, generating the annotation mask comprises receiving a user input within the GUI. For example, the user input indicates a general region of interest or an area with the disease on the image data displayed on the GUI.

In some embodiments, the one or more processors are configured to further receive a user input indicating a selection of a treatment. In some cases, the temporal sequence of images with corresponding predicted disease states are generated using an inpainting model to generate an image without the disease, and wherein the image without the disease corresponds to a final disease state. In some instances, the one or more processors are configured to further generate a first image embedding corresponding to the current disease state and a second image embedding corresponding to the final state as an outcome of the treatment. For example, the one or more processors are configured to further generate one or more temporal image embeddings corresponding to one or more disease states between the current disease state and the final state. In some cases, an encoder of the inpainting model is used as an image embedding model to generate the one or more temporal image embeddings. In some cases, the inpainting model comprises an encoder and decoder formed by gated convolution.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1 illustrates an exemplary network environment in which the systems described herein may be implemented.

FIG. 2 shows an exemplary flow chart of a method for generating temporal prediction of future state of a health condition (e.g., disease progression) and visualization of disease analytics.

FIG. 3 shows an exemplary flow chart of a method for generating temporal prediction of future disease state and disease analytics.

FIG. 4 shows an example of a network architecture of an inpainting model.

FIG. 5 shows an example of a graphical user interface (GUI) guiding a user to take an image of the user's face from multiple angles to generate 2D and 3D image representations of the user's disease state.

FIG. 6 shows an example of a GUI allowing users to provide input and learn about a disease.

FIG. 7 shows an example of a GUI for annotating an image data for a current disease state.

FIG. 8 and FIG. 9 show examples of GUIs for receiving user information.

FIG. 10 shows an example of a GUI for a user to select a treatment plan.

FIG. 11 and FIG. 12 show an example of GUI displaying multiple images with the predicted future disease state.

FIG. 13 shows an example of a GUI displaying one or more output images according to the number of treatments.

FIG. 14 shows an example of GUI displaying a 3D rendering of the face of a user.

FIG. 15 shows an example of a GUI showing a disease state score across a treatment journey.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the scope of the present disclosure. It should be understood that various alternatives to the embodiments of the present disclosure described herein may be employed.

The term “patient,” “subject” or “user,” as used herein, generally refers to an individual having, suspected of having or potentially having a disease, seeking a treatment or being associated with a treatment. The subject (or user) may be a patient seeking treatment, undergoing treatment or monitoring for the disease or health condition.

The subject as described herein may refer to an individual who are seeking service of treating the disease, monitoring a disease progression, early intervention based on the tracking of disease state, and/or prediction of future disease state through the provided platform. In some cases, a user may be a subject having skin condition/disease, esthetic-related conditions, and/or other biological conditions. The visualization tool and disease analytics provided by the systems herein may also be accessed by a caregiver, a therapist, physician, supervisor, operator, healthcare organization, insurance company, or any other entity that is associated with the patient/user through the provided platform. The provided methods and systems can allow for visualization of predicted future disease state, generating real-time feedback for intervention upon detection of deviation from a treatment path that is applicable to various healthcare areas (e.g., skin disease prognostics, cosmetic and dermatology, personal healthcare, lifestyle counseling, etc.). The term “disease state” as utilized herein may refer to a health condition, a state of a disease, a disease progression and the like.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Systems and methods herein may provide visualization of disease progression prediction and generate real-time actionable intervention that adapt to each individual. Systems and methods of the present disclosure may provide a platform for personalized disease progression prediction with improved accuracy. The platform or system may utilize computer vision and artificial intelligence techniques to allow for visualization of future disease state, treatment outcomes over a course of selected treatment, and various other analytics result. The platform or system can be used to help users effectively engage in a treatment path or program remotely. In some cases, image data captured by a user device camera (e.g., phone camera, webcam, etc.) may be required for the future state visualization and disease analytics.

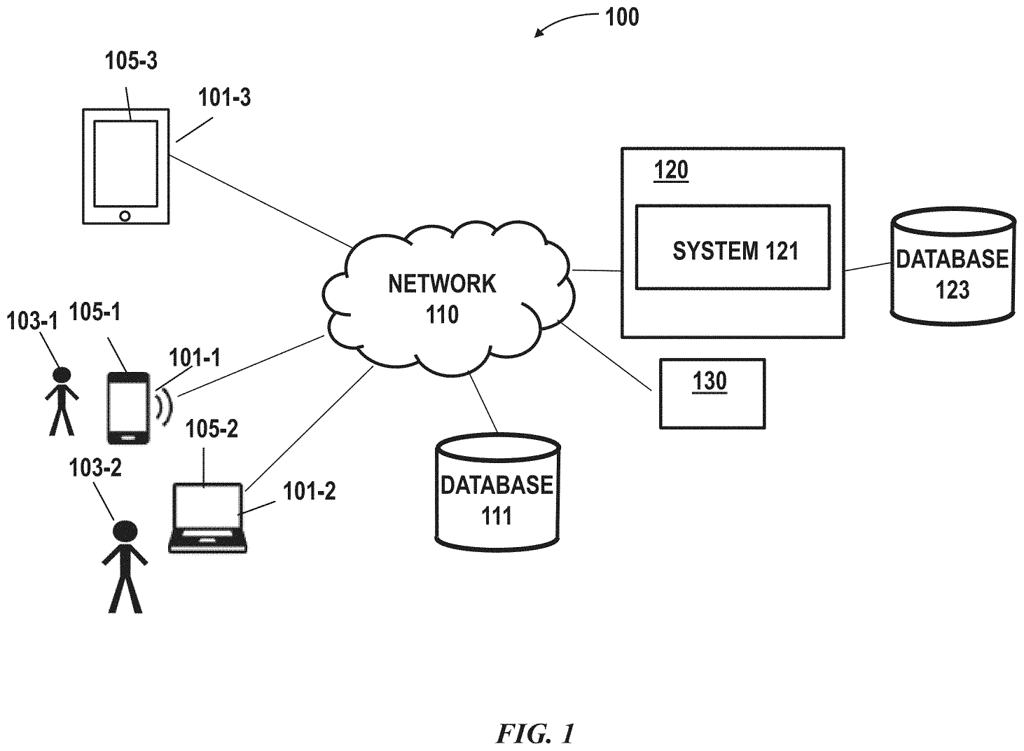

FIG. 1 illustrates an exemplary network environment 100 in which the system 121 described herein may be implemented. The network environment 100 may include one or more user devices 101-1, 101-2, 101-3, a server 120, a system 121, and a database 111, 123. Each of the components 101-1, 101-2, 101-3, 111, 123, 120 may be operatively connected to one another via network 110 or any type of communication links that allows transmission of data from one component to another.

The system 121 may be configured to analyze input data (e.g., image data) from the user device to predict one or more future disease states of a body part, generate disease analytics, and/or provide visualization of the future disease states and feedback information (e.g., guidance, disease state quantification, actionable recommendation). In some cases, the system 121 may also receive user information from the user device or from external data sources for inferring the disease states and generating the various analytics results.

The system 121 may be implemented anywhere within the network environment 100. In other embodiments, a portion of the system 121 may be implemented on the user device. Additionally, a portion of the system may be implemented on the server or cloud 120. Alternatively, the system may be implemented in one or more databases. The system may be implemented using software, hardware, or a combination of software and hardware in one or more of the above-mentioned components within the network environment.

The user device 101-1, 101-2, 101-3 may comprise an imaging sensor 105-1, 105-2, 105-3 serves as imaging device. The imaging device may be on-board the user device. The imaging device can include hardware and/or software elements. In some embodiments, the imaging device may be a camera or imaging sensor operably coupled to the user device. In some alternative embodiments, the imaging device may be located external to the user device, and image data of at least a body part of the user may be transmitted to the user device via communication means as described elsewhere herein. The imaging device can be controlled by an application/software configured to take image or video of the user. In some cases, the camera may be configured to take an image of at least a body part of the user. In some embodiments, the software and/or applications may be configured to control the camera on the user device to take image or video (e.g., live images).

The imaging device 105-1, 105-2, 105-3 may be a fixed lens or auto focus lens camera. A camera can be a movie or video camera that captures dynamic image data (e.g., video). A camera can be a still camera that captures static images (e.g., photographs). A camera may capture both dynamic image data and static images. A camera may switch between capturing dynamic image data and static images. Although certain embodiments provided herein are described in the context of cameras, it shall be understood that the present disclosure can be applied to any suitable imaging device, and any description herein relating to cameras can also be applied to any suitable imaging device, and any description herein relating to cameras can also be applied to other types of imaging devices. The camera may comprise optical elements (e.g., lens, mirrors, filters, etc.). The camera may capture color images (e.g., RGB images), greyscale image, infrared images, depth image, and the like.

The imaging device 105-1, 105-2, 105-3 may be a camera used to capture visual images of at least part of the human body. Any other type of sensor may be used, such as an infra-red sensor that may be used to capture thermal images of the human body. The imaging sensor may collect information anywhere along the electromagnetic spectrum, and may generate corresponding images accordingly.

In some embodiments, the imaging device 105-1, 105-2, 105-3 may be capable of operation at a fairly high resolution. The imaging sensor may have a resolution of greater than or equal to about 100 μm, 50 μm, 10 μm, 5 μm, 2 μm, 1 μm, 0.5 μm, 0.1 μm, 0.05 μm, 0.01 μm, 0.005 μm, 0.001 μm, 0.0005 μm, or 0.0001 μm. The image sensor may be capable of collecting 4K (3840×2160 pixels) or higher resolution images.

The imaging device 105-1, 105-2, 105-3 may capture an image frame or a sequence of image frames at a specific image resolution. In some embodiments, the image frame resolution may be defined by the number of pixels in a frame. In some embodiments, the image resolution may be greater than or equal to about 352×420 pixels, 480×320 pixels, 720×480 pixels, 1280×720 pixels, 1440×1080 pixels, 1920×1080 pixels, 2048×1080 pixels, 3840×2160 pixels, 4096×2160 pixels, 7680×4320 pixels, or 15360×8640 pixels.

The imaging device 105-1, 105-2, 105-3 may capture a sequence of image frames at a specific capture rate. In some embodiments, the sequence of images may be captured at a rate less than or equal to about one image every 0.0001 seconds, 0.0002 seconds, 0.0005 seconds, 0.001 seconds, 0.002 seconds, 0.005 seconds, 0.01 seconds, 0.02 seconds, 0.05 seconds, 0.1 seconds, 0.2 seconds, 0.5 seconds, 1 second, 2 seconds, 5 seconds, or 10 seconds. In some embodiments, the capture rate may change depending on user input and/or external conditions (e.g. illumination brightness).

The imaging device 105-1, 105-2, 105-3 may be configured to obtain image data, or scan at least a body part of the user. The imaging device may or may not be a 3D camera, stereo camera or depth camera. Various techniques can be used to obtain 3D model using the 2D imaging data of the captured body part. In some cases, the imaging device may be monocular camera and images of the user may be taken from a single view/angle. In some cases, the imaging device may be monocular camera and images of the user's body may be taken from various angles may be used to reconstruct a 3D model of the body. In some cases, the imaging device may be a 3D camera. For example, a 3D camera having three components: a conventional camera, a near infrared image sensor and an infrared laser projector may be used. Infrared parts are used to calculate the distance between objects, but also to separate objects on different planes. The 3D camera lens may have a built in IR cut filter. The 3D camera may be a video camera having a frame rate up to 60 fps with a 90° FOV, moreover its lens has an IR Band Pass filter. The IR laser integrates an infrared laser diode, low power class 1, and a resonant micro-mirror. The 3D camera may utilize technology that is implemented in a depth sensor, stereo cameras, mobile devices, and any other device that may capture depth data. In some cases, depth sensing technologies use structured light or time of flight based sensing. For example, an infrared (hereinafter, also “IR”) emitter may project (e.g., emit or spray out) beams of infrared light into the body part. The projected beams of IR light may hit and reflect off objects that are located in their path (e.g., the body part). A depth sensor may capture (e.g., receive) spatial data about the surroundings of the depth sensor based on the reflected beams of IR light. In some example embodiments, the captured spatial data may be used to create (e.g., represent, model, or define) a 3D model of body part that is displayed on a display of the user device.

In some cases, the imaging device may comprise one or more sensors that can be used to perform thermal imaging. The sensors may be passive sensors. The sensors may be able to detect heat signatures of humans. In some cases, the imaging device may include an infrared (IR) camera to perform IR imaging. Any IR camera known or later developed in the art may be used. In some instances, active illumination may be employed. For example, IR illuminator may generate infrared radiation or electromagnetic radiation where wavelengths are between 700 nanometers and 1 millimeter and may flash infrared light to assist in acquiring IR images with adequate quality. IR images may be used in conjunction with or instead of visible spectra images. Any functions provided elsewhere herein using cameras may also apply to IR cameras. Any suitable optical sensor that is sensitive to IR light may be utilized. For example, the optical sensor may use Indium Gallium Arsenide (InGaAs) focal plane array (FPA) technology and the optical sensor may have a variety of formats such as 320×256, 640×512, and 1280×1024 pixels.

User device 101-1, 101-2, 101-3 may comprise one or more imaging devices for capturing image data of one or more users 103-1, 103-2 co-located with the user device. The captured image data may then be analyzed by the system 121 to generate future disease states and analytics. In some cases, the image data may be 2D/3D image data or video data. The image data may be color (e.g., RGB) images or greyscale images. In some cases, the image data may be raw data captured by a user device camera without extra setup or cost. Details about using computer vision and machine learning techniques for the disease analytics and visualization are described later herein.

User device 101-1, 101-2, 101-3 may be a computing device configured to perform one or more operations consistent with the disclosed embodiments. Examples of user devices may include, but are not limited to, mobile devices, smartphones/cellphones, tablets, personal digital assistants (PDAs), laptop or notebook computers, desktop computers, media content players, television sets, video gaming station/system, virtual reality systems, augmented reality systems, microphones, or any electronic device capable of analyzing, receiving, providing or displaying certain types of feedback data (e.g., disease progress visualization, severity quantification analysis, actionable intervention, etc.) to a user. The user device may be a handheld object. The user device may be portable. The user device may be carried by a human user. In some cases, the user device may be located remotely from a human user, and the user can control the user device using wireless and/or wired communications.

User device 101-1, 101-2, 101-3 may include one or more processors that are capable of executing non-transitory computer readable media that may provide instructions for one or more operations consistent with the disclosed embodiments. The user device may include one or more memory storage devices comprising non-transitory computer readable media including code, logic, or instructions for performing the one or more operations. The user device may include software applications that allow the user device to communicate with and transfer data between server/cloud 120, the system 121, and/or database 111, 123. The user device may include a communication unit, which may permit the communications with one or more other components in or outside of the network 100. In some instances, the communication unit may include a single communication module, or multiple communication modules. In some instances, the user device may be capable of interacting with one or more components in the network 100 using a single communication link or multiple different types of communication links.

User device 101-1, 101-2, 101-3 may include a display. The display may be a screen. The display may or may not be a touchscreen. The display may be a light-emitting diode (LED) screen, OLED screen, liquid crystal display (LCD) screen, plasma screen, or any other type of screen. The display may be configured to show a user interface (UI) or a graphical user interface (GUI) rendered through an application (e.g., via an application programming interface (API) executed on the user device). The GUI may show prediction result, alert upon a prediction/detection of off-track status, visual representation of future disease states, images, charts, interactive elements relating to the treatment process, prognostic analytics, and real-time prediction and detection result. The GUI may permit a user to input user information. The user device may also be configured to display webpages and/or websites on the Internet. One or more of the webpages/websites may be hosted by server 120 and/or rendered by the system 121.

A user may navigate within the GUI through the application. For example, the user may select a link by directly touching the screen (e.g., touchscreen). The user may touch any portion of the screen by touching a point on the screen. Alternatively, the user may select a portion of an image with aid of a user interactive device (e.g., mouse, joystick, keyboard, trackball, touchpad, button, verbal commands, gesture-recognition, attitude sensor, thermal sensor, touch-capacitive sensors, or any other device). A touchscreen may be configured to detect location of the user's touch, length of touch, pressure of touch, and/or touch motion, whereby each of the aforementioned manner of touch may be indicative of a specific input command from the user.

In some cases, users may utilize the user devices to interact with the system 121 by way of one or more software applications (i.e., client software) running on and/or accessed by the user devices, wherein the user devices and the system 121 may form a client-server relationship. For example, the user devices may run dedicated mobile applications or software applications for viewing predicted future disease states in response to selection of a treatment, real-time quantification of treatment progress, interacting with a remote physician, receiving actionable intervention or providing user input.

In some cases, the client software (i.e., software applications installed on the user devices 101-1, 101-2, 101-3) may be available either as downloadable software or mobile applications for various types of computer devices. Alternatively, the client software can be implemented in a combination of one or more programming languages and markup languages for execution by various web browsers. For example, the client software can be executed in web browsers that support JavaScript and HTML rendering, such as Chrome, Mozilla Firefox, Internet Explorer, Safari, and any other compatible web browsers. The various embodiments of client software applications may be compiled for various devices, across multiple platforms, and may be optimized for their respective native platforms.

User device 101-1, 101-2, 101-3 may be configured to receive input from one or more users. A user may provide an input to the user device using an input device, for example, a keyboard, a mouse, a touch-screen panel, voice recognition and/or dictation software, AR/VR devices or any combination of the above. The user input may include user demographic information, statements, comments, questions, or answers relating to a disease or selection of a treatment for the disease, and various others as described elsewhere herein.

Server 120 may be one or more server computers configured to perform one or more operations consistent with the disclosed embodiments. In one aspect, the server may be implemented as a single computer, through which user device are able to communicate with the system and database. In some embodiments, the user device may communicate with the system directly through the network. In some embodiments, the server may embody the functionality of one or more of the systems 121. In some embodiments, one or more systems 121 may be implemented inside and/or outside of the server. For example, the systems 121 may be software and/or hardware components included with the server or remote from the server.

In some embodiments, the user device may be directly connected to the server through a separate link (not shown in FIG. 1). In certain embodiments, the server may be configured to operate as a front-end device configured to provide access to the system 121 consistent with certain disclosed embodiments. The server may, in some embodiments, utilize one or more systems 121 to analyze data from the user device to make inference, provide visualization of predicted future disease states and analytics and various other functions as described elsewhere herein. The server may also be configured to store, search, retrieve, and/or analyze data and information stored in one or more of the databases. The data and information may include raw data collected from imaging device on the user device, as well as each a user's historical data pattern, disease progression metrics, medical record and user provided information. In some cases, one or more predictive models may be built, developed and trained on a cloud/or the server 120. Alternatively or additionally, the one or more predictive models may be built, developed and trained on a cloud or a separate entity and run on the server 120. While FIG. 1 illustrates the server as a single server, in some embodiments, multiple devices may implement the functionality associated with a server.

A server may include a web server, an enterprise server, or any other type of computer server, and can be computer programmed to accept requests (e.g., HTTP, or other protocols that can initiate data transmission) from a computing device (e.g., user device and/or wearable device) and to serve the computing device with requested data. In addition, a server can be a broadcasting facility, such as free-to-air, cable, satellite, and other broadcasting facility, for distributing data. A server may also be a server in a data network (e.g., a cloud computing network).

A server may include known computing components, such as one or more processors (e.g., central processing units (CPUs), general purpose graphics processing units (GPUs), Tensor Processing Unit (TPU), etc.), one or more memory devices storing software instructions executed by the processor(s), and data. A server can have one or more processors and at least one memory for storing program instructions. The processor(s) can be a single or multiple microprocessors, field programmable gate arrays (FPGAs), or digital signal processors (DSPs) capable of executing particular sets of instructions. Computer-readable instructions can be stored on a tangible non-transitory computer-readable medium, such as a hard disk, a CD-ROM (compact disk-read only memory), and MO (magneto-optical), a DVD-ROM (digital versatile disk-read only memory), a DVD RAM (digital versatile disk-random access memory), or a semiconductor memory. Alternatively, the methods can be implemented in hardware components or combinations of hardware and software such as, for example, ASICs, special purpose computers, or general purpose computers. In some cases, the server or computing system may be GPU-powered servers that may each include a plurality of GPUs, PCIe switches, and/or CPUs, interconnected with high-speed interconnects such as NVLink and PCIe connections.

While FIG. 1 illustrates the server as a single server, in some embodiments, multiple devices may implement the functionality associated with server.

Network 110 may be a network that is configured to provide communication between the various components illustrated in FIG. 1. The network may be implemented, in some embodiments, as one or more networks that connect devices and/or components in the network layout for allowing communication between them. For example, user device 101-1, 101-2, 101-3, and the system 121 may be in operable communication with one another over network 110. Direct communications may be provided between two or more of the above components. The direct communications may occur without requiring any intermediary device or network. Indirect communications may be provided between two or more of the above components. The indirect communications may occur with aid of one or more intermediary device or network. For instance, indirect communications may utilize a telecommunications network. Indirect communications may be performed with aid of one or more router, communication tower, satellite, or any other intermediary device or network. Examples of types of communications may include, but are not limited to: communications via the Internet, Local Area Networks (LANs), Wide Area Networks (WANs), Bluetooth, Near Field Communication (NFC) technologies, networks based on mobile data protocols such as General Packet Radio Services (GPRS), GSM, Enhanced Data GSM Environment (EDGE), 3G, 4G, 5G or Long Term Evolution (LTE) protocols, Infra-Red (IR) communication technologies, and/or Wi-Fi, and may be wireless, wired, or a combination thereof. In some embodiments, the network may be implemented using cell and/or pager networks, satellite, licensed radio, or a combination of licensed and unlicensed radio. The network may be wireless, wired, or a combination thereof.

User device 101-1, 101-2, 101-3, server 120, and/or the system 121 may be connected or interconnected to one or more databases 111, 123. The databases may be one or more memory devices configured to store data. Additionally, the databases may also, in some embodiments, be implemented as a computer system with a storage device. In one aspect, the databases may be used by components of the network layout to perform one or more operations consistent with the disclosed embodiments. One or more local databases, and cloud databases of the platform may utilize any suitable database techniques. For instance, structured query language (SQL) or “NoSQL” database may be utilized for storing the image data, user data, historical data, predictive model or algorithms. Some of the databases may be implemented using various standard data-structures, such as an array, hash, (linked) list, struct, structured text file (e.g., XML), table, JavaScript Object Notation (JSON), NOSQL and/or the like. Such data-structures may be stored in memory and/or in (structured) files. In another alternative, an object-oriented database may be used. Object databases can include a number of object collections that are grouped and/or linked together by common attributes; they may be related to other object collections by some common attributes. Object-oriented databases perform similarly to relational databases with the exception that objects are not just pieces of data but may have other types of functionality encapsulated within a given object. In some embodiments, the database may include a graph database that uses graph structures for semantic queries with nodes, edges and properties to represent and store data. If the database of the present invention is implemented as a data-structure, the use of the database of the present invention may be integrated into another component such as the component of the present invention. Also, the database may be implemented as a mix of data structures, objects, and relational structures. Databases may be consolidated and/or distributed in variations through standard data processing techniques. Portions of databases, e.g., tables, may be exported and/or imported and thus decentralized and/or integrated.

In some embodiments, the system 121 may construct the database for fast and efficient data retrieval, query and delivery. For example, the system may provide customized algorithms to extract, transform, and load (ETL) the data. In some embodiments, the system may construct the databases using proprietary database architecture or data structures to provide an efficient database model that is adapted to large scale databases, is easily scalable, is efficient in query and data retrieval, or has reduced memory requirements in comparison to using other data structures.

In one embodiment, the databases may comprise storage containing a variety of data consistent with disclosed embodiments. For example, the databases may store, for example, raw data collected by the imaging device located on user device. The databases may also store user information, historical data patterns, data relating to a treatment progress, medical records, analytics, user input, predictive models (e.g., parameters, hyper-parameters, model architecture, threshold, rules, etc), data generated by a predictive model (e.g., intermediary results, output of a model, latent features, input and output of a component of the model system, etc.), algorithms, training datasets (e.g., image, video clips), and the like.

In certain embodiments, one or more of the databases may be co-located with the server, may be co-located with one another on the network, or may be located separately from other devices. One of ordinary skill will recognize that the disclosed embodiments are not limited to the configuration and/or arrangement of the database(s).

Although particular computing devices are illustrated and networks described, it is to be appreciated and understood that other computing devices and networks can be utilized without departing from the spirit and scope of the embodiments described herein. In addition, one or more components of the network layout may be interconnected in a variety of ways, and may in some embodiments be directly connected to, co-located with, or remote from one another, as one of ordinary skill will appreciate.

A server may access and execute system(s) to perform one or more processes consistent with the disclosed embodiments. In certain configurations, the system(s) may be software stored in memory accessible by a server (e.g., in memory local to the server or remote memory accessible over a communication link, such as the network). Thus, in certain aspects, the system(s) may be implemented as one or more computers, as software stored on a memory device accessible by the server, or a combination thereof. For example, one system(s) may be a computer training, building, developing one or more predictive models, and another system(s) may be software that, when executed by a server, performs inferences using the trained models.

The system 121 though is shown to be hosted on the server or cloud 120. The physio rehabilitation system may be implemented as a hardware accelerator, software executable by a processor and various others. In some embodiments, one or more systems 121, methods or components of the present disclosure are implemented as a containerized application (e.g., application container or service containers). The application container provides tooling for applications and batch processing such as web servers with Python or Ruby, JVMs, or even Hadoop or HPC tooling. Application containers are what developers are trying to move into production or onto a cluster to meet the needs of the business. Methods and systems of the invention will be described with reference to embodiments where container-based virtualization (containers) is used. The methods and systems can be implemented in application provided by any type of systems (e.g., containerized application, unikernel adapted application, operating-system-level virtualization or machine level virtualization).

Although various embodiments are described herein using skin condition or skin disease as an example, it should be noted that the disclosure is not limited thereto, and can be used to other types of healthcare, wellness exercising, training and activities besides medical and cosmetic purposes.

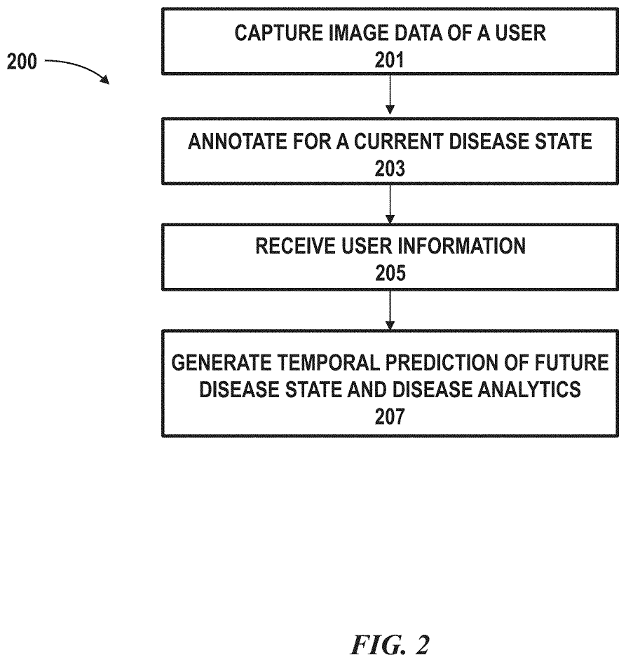

FIG. 2 shows an example of a method 200 for generating temporal prediction of future state of a health condition (e.g., disease progression) and visualization of disease analytics. The method may comprise capturing image data of a user (step 201), annotating the image data for a current state of a health condition (e.g., current disease state) (step 203), receiving user information (step 205), and generating temporal prediction of future disease state and disease analytics (step 207).

In some embodiments, capturing image data of a user (step 201) may be performed using a user device. For example, a user device (e.g., mobile device, computing device, etc.) comprising an imaging device may be used to capture the image data. The user device can be the same as those described in FIG. 1. In some embodiments, the image data maybe static image data (e.g., photographs) or dynamic image data (e.g., video, “live” image). The image data may include a visible light image, IR image, or other type of image. In some embodiments, a photo of at least a body part of the user, such as a facial portion, arm, other any body part having a disease may be acquired.

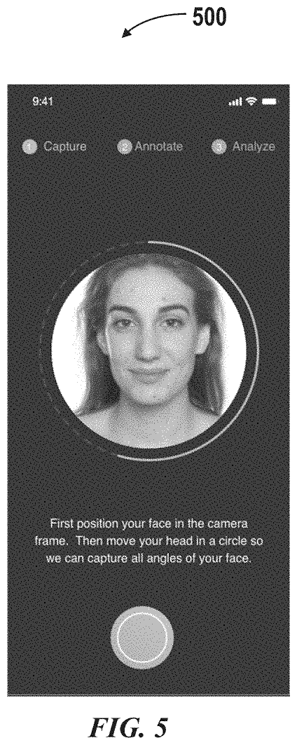

In some cases, a user may be prompted to take the image data. The image data may be 2D image. In some cases, the image data may be taken from multiple views or angles to reconstruct a 3D model of the body part. For example, a 3D reconstruction algorithm may be employed to create a robust 3D representation of the user's face. In some cases, a user may be notified if the selfie-image is not valid for further processing. FIG. 5 shows an example of a GUI 500 guiding a user to take an image of the user's face from multiple angles. For example, a user may be guided into taking an image of a body part from various positions or orientations. In some cases, an indicator indicates a successful capturing of an image may be displayed on the user device. In some cases, a user may be notified regarding the specific reason why the image is invalid (e.g., image quality is not good enough to detect a face or skin condition, insufficient views, etc.). Alternatively, a user may not be notified of the validity of the captured image.

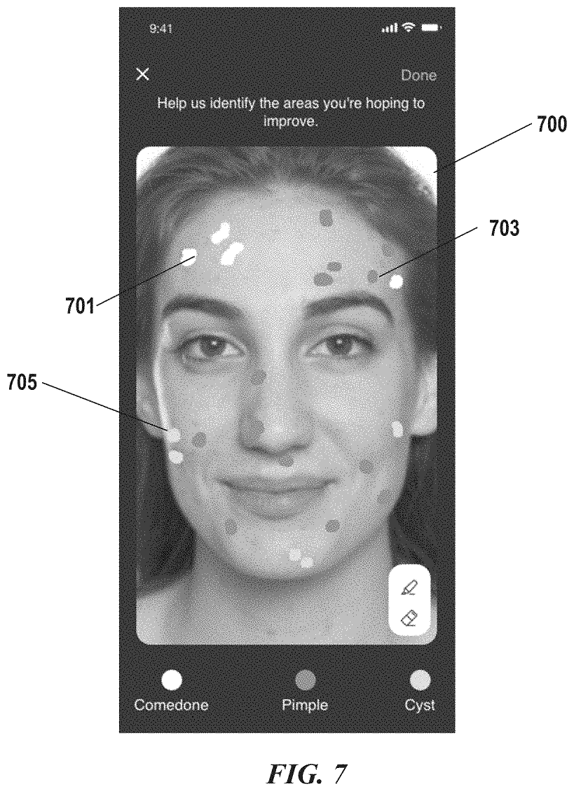

Referring back to FIG. 2, the operation of annotating the captured image for a current disease state (step 203) may be performed manually by the user, automatically using a trained algorithm or a combination of both. In some cases, a user may be prompted to annotate the image via a graphical user interface (GUI) provided by the system herein. FIG. 7 shows an example of a GUI for annotating the image data for a current disease state. The GUI may display a 2D image 700 for the user to annotate indicating a current disease state. For example, a user may touch/click on an area of the image indicating the areas they wish to improve and/or indicating a type of condition (e.g., comedone 701, pimple 703, cyst 705). In the illustrated example, the user may annotate different conditions using different colors (e.g., white for comedone 701, red for pimple 703, green for cyst 705).

In some cases, the system may generate an annotation mask based on the input, e.g., area(s) annotated by the user. In some cases, image processing algorithms (e.g., image segmentation) may be performed to generate the annotation mask of the disease or the current medical condition. For example, users' input such as touching on a location of the image (e.g., touching on the facial image indicating pimple) or drawings boundaries indicating a general region of interest may be used to generate masked regions such as by applying random dilation, rotation, cropping, or any other suitable methods. The mask generation algorithms employed by the system may be suitable for generating free-form masks or irregular masks. For example, the algorithms may follow user's sketch input and may simulate the users' behavior (e.g., touching on acne, drawings boundary of a region, masking out undesired feature, etc.).

The 2D image 700 displayed within the GUI may be generated using the captured image data. In some cases, the 2D image 700 may be the original/raw image captured by the user device. In some cases, the 2D image 700 may be generated by projecting the constructed 3D model into a 2D plane. In some cases, one or more 2D images from different angles/views (e.g., front view, left view, right view, etc.) may be displayed for the user to annotate the body part. In some cases, the 2D images of the various views (projection of the 3D model) may be displayed sequentially. User annotation inputted via previous views may be overlaid onto the later views in one or more overlapping regions. Alternatively or additionally, a user may provide annotation on a 3D model of the body part. The GUI may permit users to select a specific view (e.g., select a 2D image of a view) or interact with a 3D model for annotation.

In some cases, annotation may be performed with a trained model without requiring user input. For example, a segmentation algorithm may be applied to the input image to automatically predict a current disease state. The output of the process may be an annotation mask of the areas with the disease or a specific health condition. In some cases, the segmentation algorithm may include machine learning algorithm such as: a neural network, convolutional neural network CNN, recurrent neural network RNN, a region-based CNN (R-CNN), Faster R-CNN, Mask R-CNN, for predicting a current disease state. The system may employ any suitable object detection algorithm (e.g., R-CNN, Fast R-CNN, You Only Look Once (YOLO) algorithm, or Mask R-CNN) for determining the current disease state without user input.

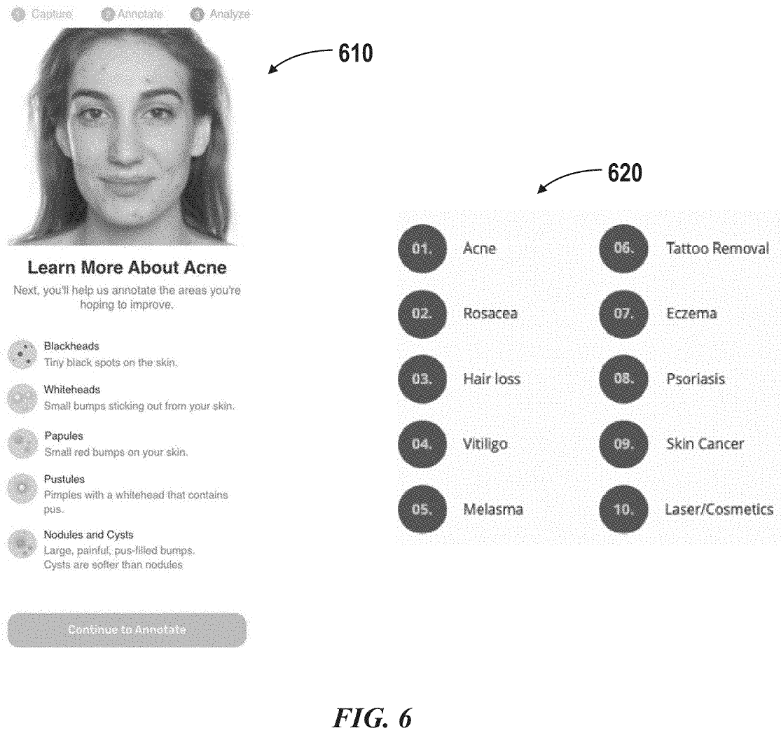

In some cases, annotation may be performed with a combination of user input and a trained model. For instance, a user may provide input indicating a general disease or a general region of interest via the GUI (e.g., draw a region of interest), and a trained model may refine the disease state such as generating an annotation mask based on the user input and the input image. For example, a user may select a region of interest and a trained model may process the image data within the region to further determine a disease state within the region of interest. FIG. 6 shows an example of a GUI permitting users to provide input about the disease. For example, the input may be simply selecting a disease. A user may choose to skip the manual annotation by selecting the disease on the GUI 620. For example, a user may select a disease from a list of diseases or health conditions displayed within the GUI 620 and the system may generate the disease masks based on the selection. In some cases, the GUI 610 may further display information educating the user about a selected disease. Such educational information along with various other features herein may beneficially increase engagement from the user.

Referring back to FIG. 2, the operation of receiving user information 205 may be performed via a graphical user interface (GUI). User information may comprise information related to demographics of the user, health history, a current treatment or past treatment the user has taken, user personal information and the like. In some cases, the user information may be requested once such as when a user first time using the application or at registration. A user may edit or modify the user information at any time point during a treatment.

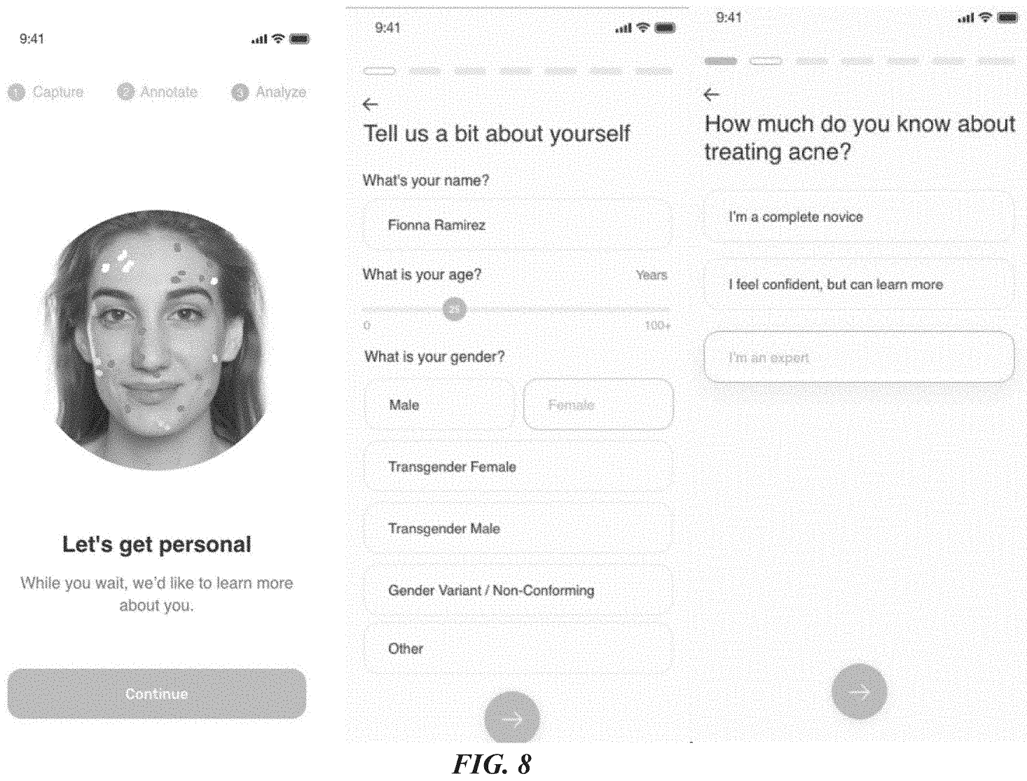

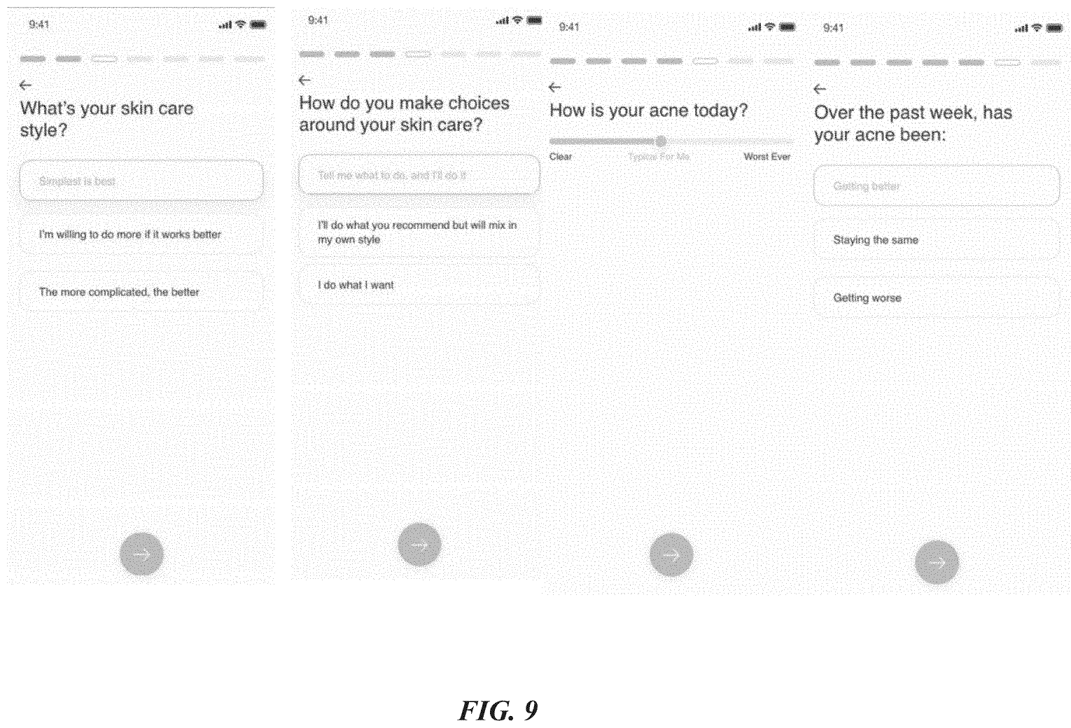

The user input may be provided by a user via the user device and/or the user application running on the user device. The user input may be in response to questions or questionnaire provided by the system. Examples of questions may be relating to a current health condition (e.g., “how is your acne today”), a current treatment or past treatment the user has taken, user personal information, and/or the like. The user's responses to those questions may be used to supplement the image data to determine the personalized disease progress prediction and visualization. FIG. 8 and FIG. 9 show examples of GUI for receiving user information data. The GUI may display one or more questions or requests for user information. In the illustrated example, the user information may be related to a user's age, gender, identity, health condition (e.g., skin care style, current skin condition), user's knowledge about the health condition, and the like. In some cases, the GUI may provide a set of options for a user to select in response to a question (e.g., “how do you make choices around your skin care”, “how is your acne today”). In some cases, the questions may be personalized based on prior user input or information about the user obtained from other data sources (e.g., medical records, etc.).

Alternatively, the GUI may permit users to provide free-form input (e.g., voice input, text input, etc.). This information obtained from the user input may be analyzed using machine learning techniques (e.g., natural language processing) and computer vision methods. For example, an NLP engine may be utilized to process the input data (e.g., input text captured from a survey, voice input, etc.) and produce a structured output including the linguistic information. The NLP engine may employ any suitable NLP techniques such as a parser to perform parsing on the input text. A parser may include instructions for syntactically, semantically, and/or lexically analyzing the text content of the user input and identifying relationships between text fragments in the user input. The parser makes use of syntactic and morphological information about individual words found in the dictionary or “lexicon” or derived through morphological processing (organized in the lexical analysis stage).

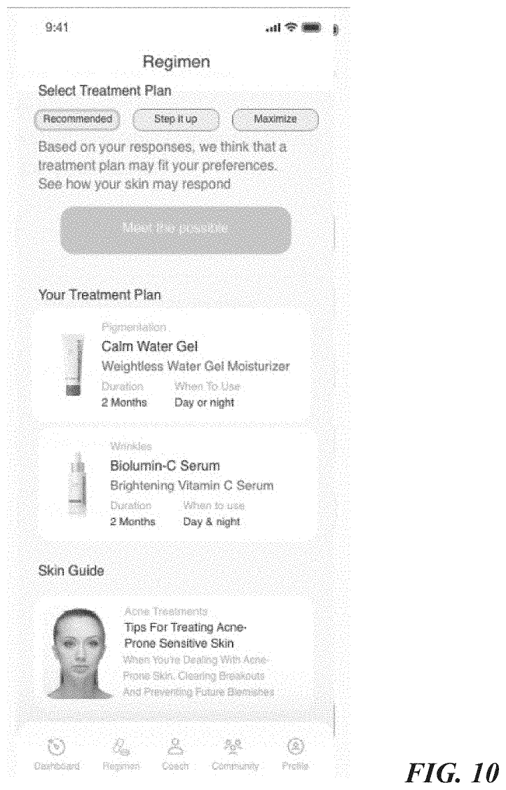

In some cases, user information may further comprise a selected treatment plan. FIG. 10 shows an example of a GUI for a user to select a treatment plan. A user may be permitted to modify or change a treatment plan to see various predicted treatment outcomes or future disease state accordingly. In the illustrated example, the GUI may display a system recommended treatment plan (e.g., “recommended”). The recommended treatment plan(s) may be generated automatically by the system based on the input image data, user information and annotation input. The GUI may also allow users to set up or customize a treatment plan, modify the recommended treatment plan and/or view multiple recommended treatment plans. The user may view the one or more recommended treatment plans, search, filter or sort the treatment plans by treatment duration, price, goals and various others based on the specific type of disease and application.

Referring back to FIG. 2, the operation for generating temporal prediction of future disease state and disease analytics 207 may be performed based at least in part on the image data, the received user information, a current disease state or annotation information. The system may employ one or more machine learning algorithm trained models for generating a series of temporal prediction of future disease states over a course of treatment.

Although the above steps show method 200 for generating temporal prediction of future state of a health condition (e.g., disease progression) and visualization of disease analytics in accordance with embodiments herein, a person of ordinary skill in the art will recognize many variations based on the teaching described herein. The steps may be completed in a different order. Steps may be added or deleted. Some of the steps may comprise sub-steps. Many of the steps may be repeated as often as beneficial to the treatment.

One or more of the steps of method 200 may be performed with processing circuitry in any one of the many devices described herein. Such circuitry may be programmed to provide one or more of the steps of the method 200, and the program may comprise program instructions stored on a computer readable memory or programmed steps of the logic circuitry.

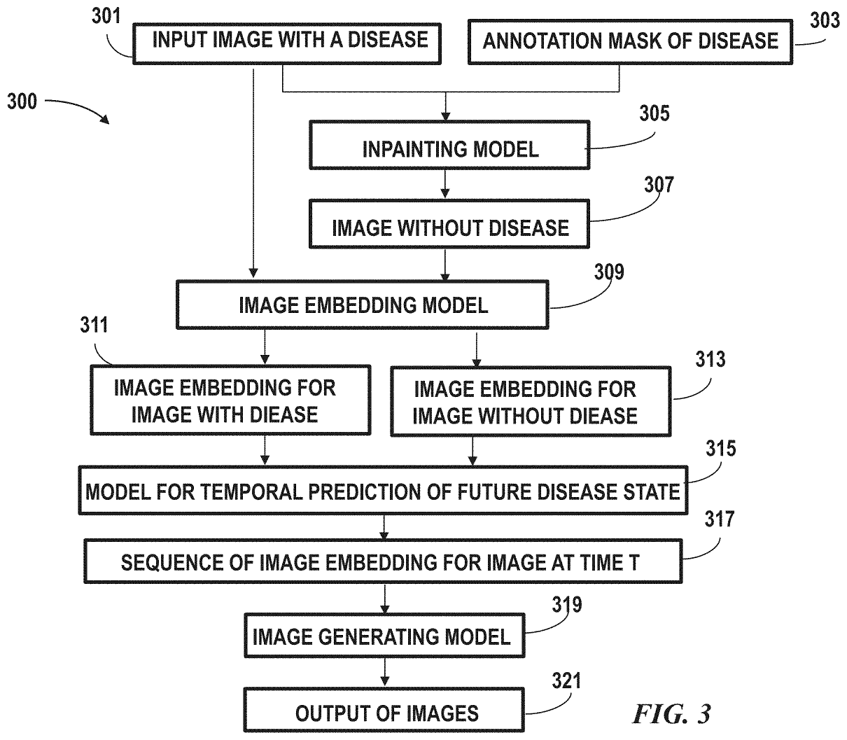

FIG. 3 shows an exemplary method 300 for generating the temporal prediction of future disease state and disease analytics. The method may employ an inpainting model 305 to process the input image data captured by the user 301 and the annotation mask of the disease 303. The input image data 301 may be the image data captured by the user indicating a disease. The input image data 301 can be the same as the image data obtained by performing the operation 201 as described in FIG. 2. In some cases, the input image data with the disease or a particular medical condition 301 may be 2D projection of 3D image/model of the user. The annotation mask of the disease 303 may be generated using the method as described in FIG. 2. The inpainting model 305 may take the input image with the disease 301 and the corresponding annotation mask of the disease 303 as input data and output an image without the disease 307 (e.g., 2D image showing a final result of a treatment or without the unhealthy area(s)).

The inpainting model 305 may perform image inpainting by synthesizing health conditions in the unhealthy regions (e.g., annotated/masked regions with disease) such that the modification is visually realistic and semantically correct. The image inpainting may be performed by, for example, patch matching the input image (with disease) using low-level image features or employing feed-forward generative models with deep convolutional networks. The inpainting model 305 herein may exploit semantics learned from large scale datasets to synthesize the contents in the masked regions. In some cases, partial convolution may be used where the convolution is masked and normalized to be conditioned on pixels outside of the masked regions.

For example, the inpainting method may utilize gated convolution capable of free-form image inpainting. The method may comprise learning a dynamic feature gating mechanism for each channel and each spatial location such as inside or outside masks, or RGB channels. For instance, the input feature may be used to first compute gating values and the final output of the model may be a multiplication of learned feature and the gating values. Gated convolution can provide improved performance particularly when the masks (e.g., disease annotation masks) have arbitrary shapes and/or the inputs have conditional inputs such as sparse sketch (e.g., user input generally indicating a disease, interested body part or general region of interest). This beneficially allows for inpainting images accounting for a variety of user input/annotation information without requiring the input features limited to RGB channels with masks.

The inpainting model 305 can have any suitable network architecture. In some cases, the network architecture may include gated convolution stacked to form an encoder-decoder network. In some cases, the process of training the inpainting model may comprise extracting unsupervised features from the input data. In some cases, the input data may not include labeled data. The model network may comprise an autoencoder. During the feature extraction operation, the autoencoder may be used to learn a representation of the input data for dimensionality reduction or feature learning. The autoencoder can have any suitable architecture such as a classical neural network model (e.g., sparse autoencoder, denoising autoencoder, contractive autoencoder) or variational autoencoder (e.g., Generative Adversarial Networks (GAN)).

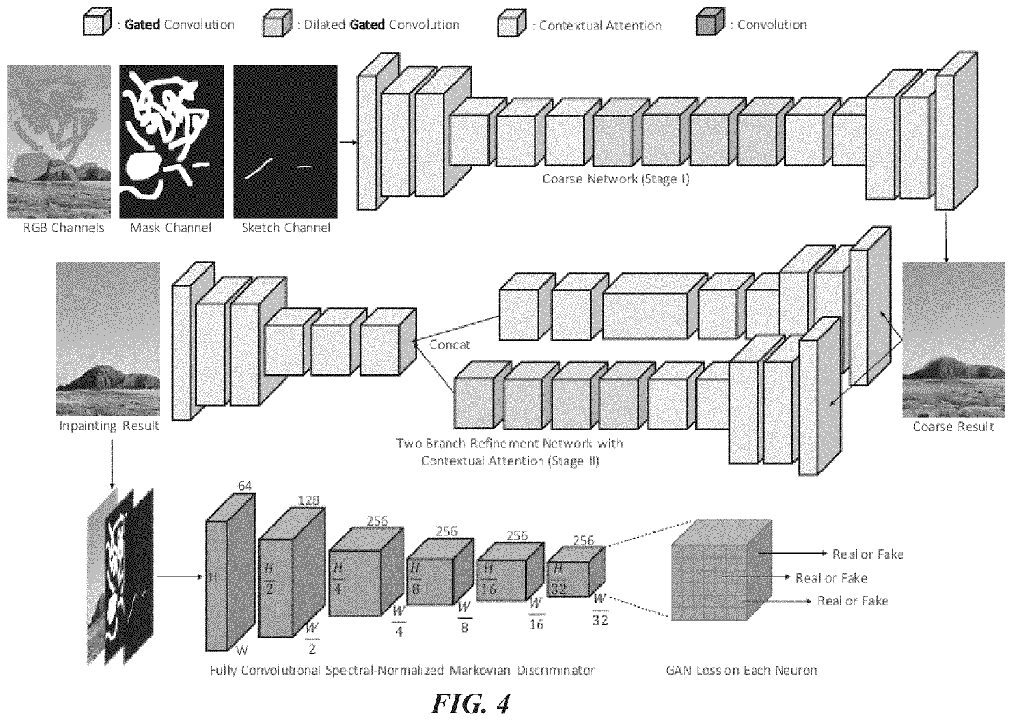

FIG. 4 shows an example of the network architecture of the inpainting model 305. In some cases, the model 305 may be trained with pixelwise reconstruction loss and adversarial loss which is suitable for free-form image inpainting. The network architecture may comprise a variant of Generative Adversarial Networks (GAN) that is fast and stable in training and capable of producing high-quality inpainting results. The discriminator of the GAN may directly compute hinge loss on each point of the output map focusing on different locations and different semantics (represented in different channels). The discriminator may be a patch-based GAN discriminator. The illustrated generative inpainting network may comprise coarse and refinement networks with encoder-decoder architecture.

It should be noted that the inpainting model 305 can have any suitable network architecture. For example, the deep learning network may employ U-Net architecture with skip-connections that forward the output of each of the encoder layers directly to the input of the corresponding decoder layers. As an example of a U-Net architecture, upsampling in the decoder is performed with a pixelshuffle layer which helps reducing gridding artifacts. The merging of the features of the encoder with those of the decoder is performed with pixel-wise addition operation resulting in a reduction of memory requirements. The residual connection between the central input frame and the output is introduced to accelerate the training process. In another example, the deep learning network may include a sparse autoencoder with an RNN (recurrent neural network) architecture, such as LSTM (long-short-term memory) network, trained to regenerate the inputs for dimensionality reduction. For example, an encoder-decoder LSTM model with encoder and decoder layers may be used to recreate a low-dimensional representation of the input data to the following model training despite a latent/hidden layer.

The decoder of the inpainting model may be trained to recover feature map resolution. For example, the decoder network of the inpainting model may take as input the encoder produced output and recover the image from it that remains visually similar to input image of the encoder.

Referring back to FIG. 3, the output of the inpainting model may be an image without the disease 307. The image without disease 307 may be generated by the decoder as described above which may have same resolution of the input image with the disease and remain visual similarity. The image without disease 307 and the input image with the disease 301 may be inputted to an image embedding model 309 and the output of the image embedding model may comprise image embedding for the image with the disease 311 and the image embedding for the image without disease 313. An image embedding is the low-dimensional, learned continuous vector representations of the input image. For example, the image embedding for the image with disease 311 may be a low-dimensional representation of the input image with the disease 301 and the image embedding for the image without disease 313 may be a low-dimensional representation of the image without disease 307.

The image embedding model 309 may utilize any suitable model to create the image embedding. In some cases, the encoder of the inpainting model may be used as the image embedding model 309. The encoder trained as part of the inpainting model 305 may take the input image with the disease 301 and the image without disease 307 (output of the inpainting model). The encoder may be trained on the training datasets same as those used for training the inpainting model 305. For example, the low-dimensional representation i.e., image embedding, may be a vector of continuous numbers mapped from the input image by the encoder. The image embedding model can be any other models and the image embedding may be different accordingly. For example, the image embedding model and image generating model 319 may be an identity function such that the output image and the input image may have the same dimensionality.

The image embedding for the image with disease 311 and the image embedding for the image without disease 313 may then be processed by the model 315 which is configured for generating temporal prediction of future disease state. The output of the model 315 may be a sequence of image embeddings for the input image at a sequence of future time points. The sequence of future time points T1, T2, . . . Tn may represent the time points over a course of treatment. The initial time point of the treatment may correspond to the disease state represented by the image with the disease 301 and the last time point of the treatment may correspond to the disease state represented by the image without the disease 307. The model 315 may be trained to predict the transition or midpoints across the treatment based on the input initial image embedding (image embedding for the image with disease 311) and the final image embedding (image embedding for the image without disease 313). The model 315 may predict the future disease state corresponding to one or more future time points from the initial to the final time point. The time points may be predicted based at least in part on the received user information (e.g., demographic information, selected treatment plan, etc.). In some cases, the model 315 may iteratively predict or update a “next state” based on currently available information.

In some cases, a model 315 may only takes an embedding for the image with disease, and iteratively update the embedding and/or predict multiple future time point embeddings simultaneously without knowing the final disease state (i.e., no disease) embedding in advance.

The model 315 for predicting the future disease states (e.g., image embedding with the future disease state corresponding to various time points) may employ any suitable methods/algorithms. For example, the model may be a deep learning network. In some cases, the predicted future disease states may be worse than an initial condition (e.g., predicting more disease). For example, when taking acne medication, the acne may get worse initially before reducing the symptom. The model may predict additional acne lesions in the first one or more time points and subsequently predict fewer acne lesions in later time points. Alternatively, the model may be a linear model trained to the interpolate the intermediate disease states over a course of treatment.

Next, the temporal sequence of image embedding corresponding to the temporal sequence of predicted disease states 317 may be processed by an image generating model 319 to output the images 321. The output images may be displayed to the user on a GUI.

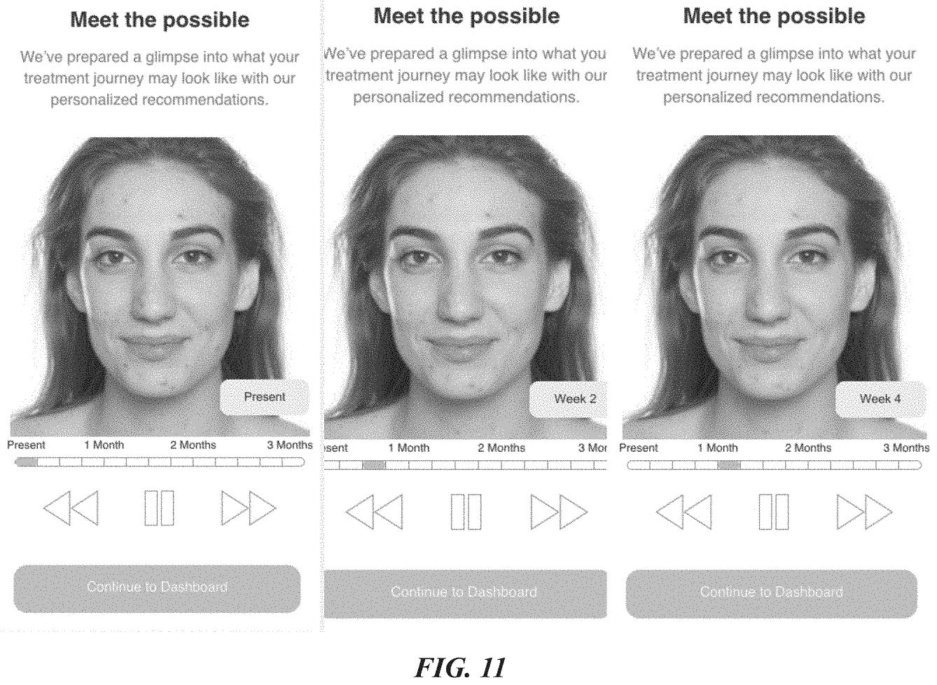

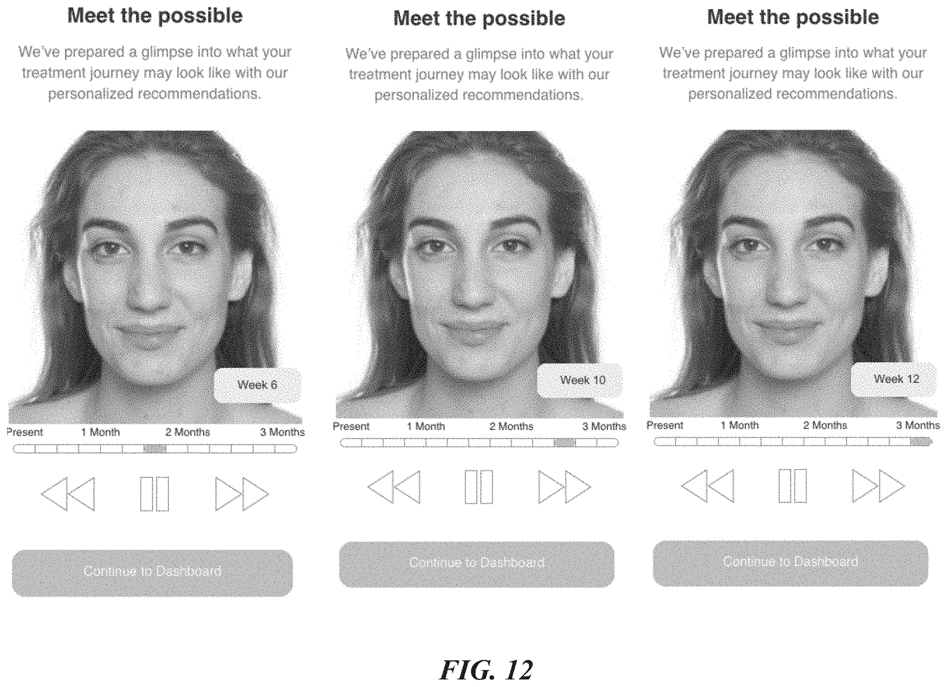

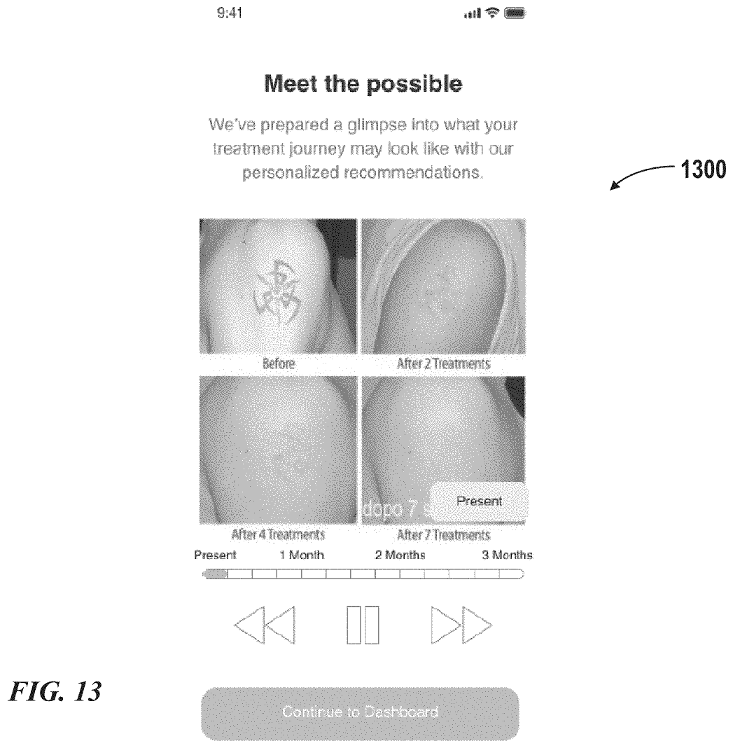

FIG. 11 and FIG. 12 show an example of GUI displaying multiple images with the predicted future disease state. A user may select a time point (e.g., week 2, week 4, week 6, 2 months, etc.) to view the corresponding image and disease state. The GUI may also allow users to go backward and forward to view a previous state or a next state. The time points may be automatically predicted by the model 315. In some cases, the time points may be evenly distributed across the course of treatment. In some cases, the time points may be predicted transition point where a visual change can be perceived. In some cases, the time points may be based on the treatment phase (e.g., number of treatments). FIG. 13 shows an example of the GUI 1300 displaying the output images according to the number of treatments.

The output images (such as shown in FIG. 11 and FIG. 12) may be synthesized input image with the predicted future disease states. Referring back to FIG. 3, the image generating model 319 can include any suitable model. For example, the image generating model 319 may be the decoder (for recovering feature map resolution) trained as part of the inpainting model 305. The decoder may generate the output image based on the sequence of image embedding at the future time point where the output image may have the same resolution of the input image with the disease 301. Any other suitable models may be utilized for generating the output images. For example, the image embedding model and the image generating model may be an identity function approximated by the autoencoder of the inpainting model.

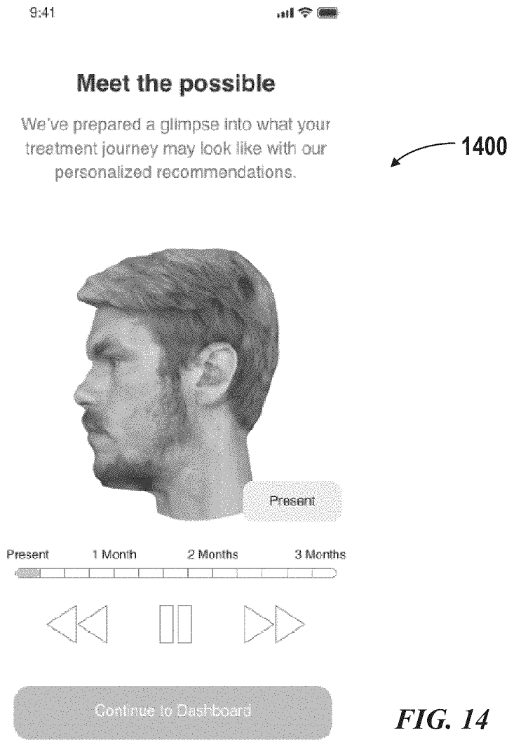

In some embodiments, the output image may be a 3D rendering of the body part generated based on the input image data. FIG. 14 shows an example of GUI displaying a 3D rendering of the face of a user 1400. A user may select different time points to view the image from different views/orientations by interacting with the 3D model.

In some embodiments, the input image with the disease may be a 3D image or thermal image. Such input image data can be processed in a similar manner using the method as described in FIG. 3. For example, the same method as described in FIG. 3 may be applied to the 3D image by first projecting the 3D image into 2D image planes, predicting the future disease states (e.g., image embedding at the various time points as described above) and reconstructing a 3D output image using the 2D images with the predicted disease states.

Although the above steps show method 300 for generating the temporal prediction of future disease state and disease analytics in accordance with embodiments herein, a person of ordinary skill in the art will recognize many variations based on the teaching described herein. The steps may be completed in a different order. Steps may be added or deleted. Some of the steps may comprise sub-steps. Many of the steps may be repeated as often as beneficial to the treatment.

One or more of the steps of method 300 may be performed with processing circuitry in any one of the many devices described herein. Such circuitry may be programmed to provide one or more of the steps of the method 300, and the program may comprise program instructions stored on a computer readable memory or programmed steps of the logic circuitry.

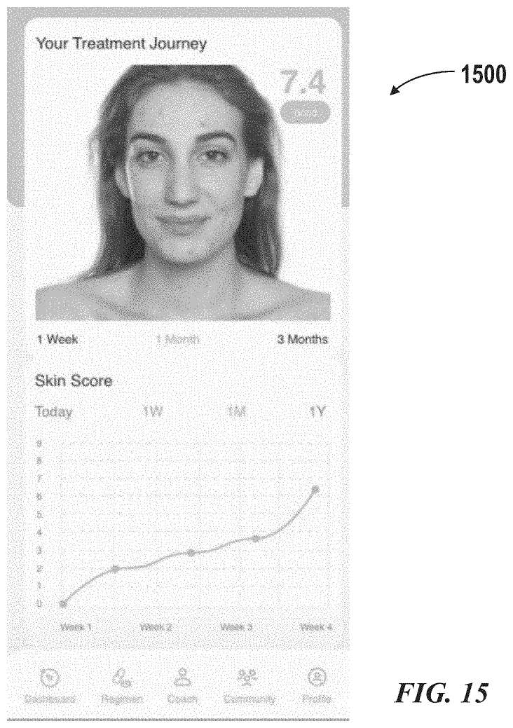

Referring back to FIG. 2, the system may also generate various analytics relating to the disease progression 207. In some embodiments, the system may quantify the disease state such as by producing a score indicating the severity of the disease progression. FIG. 15 shows an example of a GUI 1500 showing the score across a treatment journey. The score may quantitively represent the treatment progress or disease severity. The score may be generated using a trained model or any other suitable algorithms. In some cases, the score may be calculated based on a comparison with a group of patients/users with the same disease.

In some cases, the GUI may also display a plot of the treatment journey timeline or score timeline with error bars showing the projection of how the user will do in the future. In some cases, the treatment journey timeline may also illustrate the causal relationship between user's actions (e.g., missing a treatment, modifying a treatment such as timing or dose, delaying a treatment action item, etc.) and the score, and visually illustrate the impact of such actions on the score. For instance, the score timeline may allow users (e.g., patients) to visualize the impact that actions in the past have had on the present. For instance, the score timeline may model or simulate different actions and the impact to help users understand the difference. For example, a user may be provided a simulated result of missing a treatment and may allow the user to visualize the impact of the behavior on future treatment progress (e.g., score timeline). For instance, a user may interact with the score timeline GUI to visualize missing a treatment will result in decrease of the score in the future time.

In some cases, the GUI may also display one or more suggested action items for remaining or getting back on track of the treatment progress. A user may be provided with actionable feedback (e.g., intervention) to help the user improve the score or stay on track of the treatment path. For example, the system may track (e.g., track the skin condition of the user by requesting images from the user) the treatment progress and when it is determined to be not on-track (e.g., a deviation beyond a pre-determined threshold), the system may deliver feedback to the user with actionable suggestions. In some cases, a real therapist may intervene and may contact the patient. In some cases, the feedback may be delivered through the user device or the application running on the user device. The feedback may comprise, for example, detected deviation from the treatment path, warning of an incorrect or incomplete treatment, suggestion or recommendation of correcting a current medical condition, and various others. The feedback may be delivered to the user through any suitable communication channel and/or through the user device.

In some embodiments, the system may track and update of the disease analytics over time. For example, a user may modify a treatment or user information during the course of treatment. Upon receiving such updates, the system may automatically repeat the operations described in FIG. 2 and FIG. 3 to update the disease analytics and predicted future disease states.

In some cases, one or more models employed by the system (e.g., encoder for extracting unsupervised features, inpainting model, annotation mask generation model, etc.) may be refined or tuned as new data is collected (e.g., new image data). For example, a pre-trained sparse autoencoder for unsupervised feature extraction may undergo additional training as it attempts to regenerate the input data collected by the user device. This is beneficial to account for variability in implementation of the system.

The training datasets for training one or more of the models may be obtained from multiple sources. For example, the training data may be obtained from public datasets containing images of people or body part (e.g., faces), clinical data, data provided by patients/users. The training data may include disease-specific data for disease progression prediction.

In some cases, the system may employ data augmentation techniques to augment the training data. The data may be augmented based on the specific disease. For example, for predicting disease states associated with acne, smaller cropping may be used for each acne patch, and as the disease progression for acne can be modeled in a much more local manner. For instance, a user has 20 individual acne lesions, 20 images from a single image of the face may be cropped, and augmentation methods (e.g., random perturbations to pixels, random image rotations and horizontal/vertical mirroring, random resizing and random cropping of images, etc.) may be applied to each acne lesion cropping.

The various functions performed supported by the client terminal such as disease analytics generation, visualization, continual training, data processing, executing a trained model and the like may be implemented in software, hardware, firmware, embedded hardware, standalone hardware, application specific-hardware, or any combination of these. The system, components of the systems, and techniques described herein may be realized in digital electronic circuitry, integrated circuitry, specially designed ASICs (application specific integrated circuits), computer hardware, firmware, software, and/or combinations thereof. These systems, devices, and techniques may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. These computer programs (also known as programs, software, software applications, or code) may include machine instructions for a programmable processor, and may be implemented in a high-level procedural and/or object-oriented programming language, and/or in assembly/machine language. As used herein, the terms “machine-readable medium” and “computer-readable medium” refer to any computer program product, apparatus, and/or device (such as magnetic discs, optical disks, memory, or Programmable Logic Devices (PLDs)) used to provide machine instructions and/or data to a programmable processor.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for predicting future disease states of a subject, comprising:
(a) obtaining an image from an imaging device, wherein the image indicates a disease associated with the subject;
(b) generating an annotation mask for a current disease state;
(c) processing the image data and the annotation mask to generate a temporal sequence of images with corresponding predicted disease states; and
(d) outputting the temporal sequence of images and disease analytics within a graphical user interface (GUI).

2. The method of claim 1, wherein the disease analytics comprise scores of the future disease states over a course of treatment.

3. The method of claim 1, wherein generating the annotation mask comprises receiving a user input within the GUI.

4. The method of claim 3, wherein the user input indicates a general region of interest or an area with the disease on the image data displayed on the GUI.

5. The method of claim 1, further comprising receiving a user input indicating a selection of a treatment.

6. The method of claim 5, wherein generating the temporal sequence of images with corresponding predicted disease states comprises using an inpainting model to generate an image without the disease, and wherein the image without the disease corresponds to a final disease state.

7. The method of claim 6, further comprising generating a first image embedding corresponding to the current disease state and a second image embedding corresponding to the final state as an outcome of the treatment.

8. The method of claim 7, further comprising generating one or more temporal image embeddings corresponding to one or more disease states between the current disease state and the final state.

9. The method of claim 8, wherein an encoder of the inpainting model is used as an image embedding model to generate the one or more temporal image embeddings.

10. The method of claim 6, wherein the inpainting model comprises an encoder and decoder formed by gated convolution.

11. A system for predicting future disease states of a subject, the system comprising:
(i) a memory for storing a set of software instructions, and (ii) one or more processors configured to execute the set of software instructions to:
(a) receive an image acquired by an imaging device, wherein the image indicates a disease associated with the subject;
(b) generate an annotation mask for a current disease state;
(c) process the image data and the annotation mask to generate a temporal sequence of images with corresponding predicted disease states; and
(d) output the temporal sequence of images and disease analytics within a graphical user interface (GUI).

12. The system of claim 11, wherein the disease analytics comprise scores of the future disease states over a course of treatment.

13. The system of claim 11, wherein generating the annotation mask comprises receiving a user input within the GUI.

14. The system of claim 14, wherein the user input indicates a general region of interest or an area with the disease on the image data displayed on the GUI.

15. The system of claim 11, wherein the one or more processors are configured to further receive a user input indicating a selection of a treatment.

16. The system of claim 15, wherein the temporal sequence of images with corresponding predicted disease states are generated using an inpainting model to generate an image without the disease, and wherein the image without the disease corresponds to a final disease state.

17. The system of claim 16, wherein the one or more processors are configured to further generate a first image embedding corresponding to the current disease state and a second image embedding corresponding to the final state as an outcome of the treatment.

18. The system of claim 17, wherein the one or more processors are configured to further generate one or more temporal image embeddings corresponding to one or more disease states between the current disease state and the final state.

19. The system of claim 18, wherein an encoder of the inpainting model is used as an image embedding model to generate the one or more temporal image embeddings.

20. The system of claim 16, wherein the inpainting model comprises an encoder and decoder formed by gated convolution.
